(19) United States
(12) Patent Application Publication
(10) Pub. No.: US 2012/0208222 A1
(43) Pub. Date: Aug. 16, 2012

(54) Microchemical reactor

(76) Inventors: Andrew Mark Hecht, Sandia Park, NM (US); Paul Martin Baca, Albuquerque, NM (US)

(21) Appl. No.: 12/931,793

(22) Filed: Feb. 10, 2011

(51) Int. Cl. C12Q 1/46; B23P 11/00; G01N 30/00

(52) U.S. Cl. 435/20; 422/69; 422/88; 29/428

(57) ABSTRACT

Microchemical reactors embedded in absorbant materials are described that allow operators to perform chemical reactions and chemical cascades using multiple microencapsulated reagents, which release stoichiometric amounts of reagents in a predetermined time sequence with a predetermined logic.

### BACKGROUND OF THE INVENTION

1. Field of the Invention

This invention relates to microchemical reactors employing microencapsulated reagents to facilitate chemical reactions and cascades for sensors, analytical systems and other chemical, biochemical and medical applications.

2. Description of the Prior Art

It is the objective of this invention to provide a fast, simple, inexpensive, and convenient way to perform chemical reactions for analytical devices, chemical sensors and detectors, and other applications involving chemical reactions and chemical cascades. This invention uses microencapsulated reagents where the microcapsules are designed such that a chemical reaction, once started, automatically proceeds to its completion. Prior art exists for the technology of microencapsulating chemicals, and this invention makes use of the prior art of microencapsulation. Prior art also exists for handheld, easy to use, tranportable chemical sensors and diagnostic devices for medical and environmental applications. Some examples of devices that are on the market at the time of this writing include blood glucose meters, home cholesterol tests, and point-of-care C-reactive protein tests. There is also a need for devices that can detect potentially harmful chemicals in the environment, such as pesticides and herbicides. This invention provides a new technique for making handheld devices that will allow inexpensive and easy to use chemical analysis systems and devices.

This invention further allows chemical reactions to be incorporated into every day objects. For example, bathroom tissue, cotton swabs, as well as other objects that come into contact with the human body or bodily fluids may now become a medical diagnostic, or environmental measuring device. This would allow individuals to test themselves for diseases, monitor their health, or test their environment for dangerous chemicals.

Prior art exists to incorporate a test for fecal occult blood into toilet paper. U.S. Pat. No. 5,840,584 describes a device where the fecal occult blood test is incorporated into bathroom tissue, allowing people to detect fecal occult blood in their feces. This invention, however, does not use microencapsulated reagents. It describes a sheet of brittle material which houses a plurality of chambers which hold a liquid reagent solution. It also requires absorbent material impregnated with another reagent, guiaiac material. This invention is difficult and expensive to manufacture.

Prior art exists for the mitigation of a chemical or biological compound. For example, if chemical warfare agents or bacterial spores are applied to a surface, the area needs to be decontaminated. In the case of chemical agents and biological spores that have contaminated a building structure and its contents, foams have been devised which are applied to contaminated surfaces. The foams deliver reagents that denature these chemical and biological agents, rendering them non toxic. In current practice water and surfactant are mixed with air to produce a free flowing foam. An active ingredient which denatures the chemical or biological agent is added to the foam. Because of the method by which the active ingredient is entrained in the aqueous foam, much of its effectiveness to mitigate the chemical or biological agent is lost. Another problem of the current technology is short shelf life of the active ingredients.

Prior art exists to encapsulate chemical reagents. A reagent is said to be encapsulated when it is immobilized in microcapsules ranging from a diameter of a less than a micrometer to millimeters. Micro-capsules have shells that can be made of various materials. The encapsulated reagent within the shell is not chemically affected by the encapsulation process. The micro-encapsulation process protects the reagent from the environment.

Prior art techniques exits to control microcapsule sizes and volume of contents. Those skilled in the art of microencapsulation can design porous microcapsule shell walls to allow reagents to enter and exit the microcapsule. This might be useful to allow access to a microencapsulated enzyme or catalyst. Techniques are known to produce friable microcapsules that liberate their contents when mechanically ruptured.

Prior art exists which combines microencapsulated reagents on test swabs. U.S. Pat. No. 5,278,075 describes a swab for detecting the presence of a metal on a solid surface which comprises a microencapsulated reagent that reacts with the metal. However this patent does not claim to colocate multiple microencapsulated reagents such that more than one microencapsulated reagent may partake in a chemical reaction or cascade. Additionally, this patent does not provide a support that serves as a known volume, allowing stoichiometric analytical chemistry and therefore measurements of analyte concentration. This patent also does not provide for microencapsulated process solvent.

U.S. Pat. No. 6,406,876 B1 teaches a method by which an enzyme used for the detection of organophosphate and related materials is covalently attached to a porous support. This invention does not provide a way to automate the timing of release of reagents, nor is the method of covalent attachment used in this patent as effective as microencapsulation at increasing shelf life of biomolecules, such as enzymes.

Prior art exists which automate chemical reactions using mechanical fluidics to control the flow'of reagents in chemical reactions. U.S. Pat. No. 6,861,252 teaches that a sensor for detecting an analyte in the environment can be constructed using reservoirs containing carrier fluids and activators that interact with reagents embedded in polymers. The invention requires mechanical devices, including reservoirs, valves and pumps, which make the invention complicated and cumbersome to operate.

Prior art technology used to automate chemical reactions for chemical sensors includes impregnating various carrier materials by solvent or physical deposition on porous or sponge-like supports. U.S. Pat. No. 7,491,546 teaches a method of water analysis for chlorine in which the chemistry is touch free, utilizing solvent deposited reagents on an insoluble fibrous matrix. This invention does not a provide a way to automate the release of different reagents at specific times, nor does the reagent impregnation method help to protect sensitive molecules from the environment and maximize reagent shelf life.

### SUMMARY OF THE INVENTION

Briefly described, the present invention relates to methods for making microchemical reactors that are comprised of microencapsulated reagents that can be embedded in or on objects. This invention opens new possibilities for the use of chemistry. It allows both simple and complex chemistry to be portable, automated, simplified, cost effective, and versatile.

It is therefore the primary object of the present invention to provide microencapsulated reagents in stoichiometric ratios which are embedded in objects for the purpose of making microchemical reactors.

Another object of the present invention is to colocate microcapsules containing different reagents that are embedded in objects so that chemical reactions can occur in the same location, which allows many types of easy to use devices to be created. The smallest chemical microreactor consists of two colocated 1 micron diameter microcapsules each containing a different reagent. When these reagents are released from their shells, and react, the smallest microreactor volume is 4.18 attoliters.

A further object of the present invention is to automate chemical reactions by selecting microcapsule shells of microencapsulated reagents that are either friable, solvent specific, porous, or of varying thickness. All of these techniques can be used to release the microcapsule contents at specific times and under specific conditions.

It is still a further object of the present invention to employ chemical logic, if required by the application. This allows chemical reactions in this invention to select a particular chemical pathway from multiple possible chemical pathways, based on conditions that are experienced by the shells of microencapsulated reagents.

### BRIEF DESCRIPTION OF THE DRAWINGS

Reference is now made to the accompanying drawings, which forms a part of the specification of the present invention.

FIG.1 is a side view of the present invention as the online reagent strip.

FIG.2 is a top view of the reagent strip.

FIG 3. is a perspective view of the present invention configured as a strip device for detection of pesticides and nerve agents.

FIG 4. is a perspective view of present invention as a sponge tipped swab.

FIG 5. is a perspective view of the present invention as a sponge tipped swab placed into a transparent reaction container.

FIG 6. is a plan view of the present invention configured as a section of bathroom tissue that detects fecal occult blood.

FIG 7. is a side view of the present invention configured as a section of bathroom tissue that detects fecal occult blood.

FIG 8. is block diagram of the present invention configured in a process control system.

### DETAILED DESCRIPTION OF THE INVENTION

### Definitions

Microencapsulation, as used herein, is the process of placing a spherical shell composed of a synthetic or biological polymer completely around another chemical for the purpose of delaying or slowing its release.

A microencapsulated reagent, as used herein, is any chemical that has been microencapsulated.

A microcapsule, as used herein, is a spherically shaped capsule consisting of a shell of varying thickness surrounding a core of liquid or solid chemical. The microcapsule can range in diameter from a micron to several millimeters.

Shell, as used herein, is any synthetic or biological polymer which completely surrounds the core of an encapsulated reagent.

Core, as used herein, is any chemical compound which is surrounded by a spherical shell for the purpose of encapsulation.

Analyte, as used herein, is the specific substance or chemical constituent that is being measured in a chemical analysis.

Working solvent, as used herein, is any liquid which enables the mixing and reaction of reagents to allow chemical reactions.

Foam, as used herein, is an emulsion-like two-phase system where the dispersed phase is gas or air. In the case of rigid insoluble open cell foam the dispersed phase is located within a solid resinous matrix, the cell walls of which are open to the external environment.

A stoichiometric ratio or amount of a reagent, as used herein, is the amount or ratio where, assuming the reaction proceeds to completion, all reagent is consumed, there are no shortfall of reagent and no residues remain.

A surfactant, as used herein, is a soluble compound that reduces the surface tension of liquids or reduces interfacial tension between two liquids or a liquid and a solid.

A microchemical reactor, as used herein, is a device comprised of microencapsulated chemical reagents which are placed in or on an object, which microencapsulated reagents range in size from 1 micron to 2 millimeters, and when combined with an analyte, or each other, produce a desired chemical reaction.

Microchemical reactor, as used herein, is a device that is comprised of microcapsules containing different reagents needed for a desired chemical reaction, combined in correct stoichiometric ratio. The smallest chemical microreactor consists of two colocated 1 micron diameter microcapsules each containing a different reagent. When these reagents are released from their shells, and react, the smallest possible microreactor volume is 4.18 attoliters. If a larger quantity of reaction material is desired, the mixture is extended in length, area, or volume, hence the structure of the reactor is extensible.

### The Best Mode of the Invention

The present invention is comprised of microencapsulated reagents. Five different types of shells used in microencapsulation are used to control the reaction sequence and timing. These types are 1) friable shells 2) shells of varying thickness and 3) shells that are solvent specific 4) porous shells and 5) shells that are caused to open by techniques other than mechanical rupture or dissolving, including the absorption of acoustic and electromagnetic energy.

In the present invention, the first method of automating chemical reaction sequence timing is by using a friable microencapsulated reagent, which becomes part of the reaction chain when it is mechanically ruptured. The second method of automating chemical reaction sequence timing is by selecting the thickness of the shell material, whereby, for a specific working solvent, thicker shells take more time to dissolve and release encapsulated contents. The third method of automating chemical reaction sequence timing is by selecting specific shell materials that dissolve at different rates for a specific solvent. The fourth method of automating chemical reaction sequence timing is by selecting shell materials that are porous and allow reactions to occur without releasing the shell contents. Porous shells are useful for some applications wherein it is desirable to re-use a chemical reagent, such as a catalyst or enzyme. The fifth method of automating chemical reaction sequence timing includes using electromagnetic or acoustic energy to open the shell and release the contents. Sensors can be made from microcapsules comprised of material that absorbs either electromagnetic or acoustic energy. This absorption of energy causes the shell of the microcapsule to open, indicating the presence of electromagnetic or acoustic energy. Any combination of these techniques for automating chemical reactions may be used in a system.

The present invention uses microencapsulated reagents in pre-measured quantities so that the chemical reactions are in proper stoichiometric ratios. Some microcapsules may contain more than one reagent. Some microcapsules may be comprised of nested concentric shells which contain different reagents between concentric shells.

The present invention allows two or more chemically distinct microencapsulated reagents to be colocated so that all chemical reactions can occur in the same location, which allows many types of easy to use devices to be created. This allows the reactor to be built onto surfaces or inside structures conducive to their subsequent use. The support can be designed to absorb a predetermined amount of analyte solution or mixture which may be solid, liquid, or gas.

The present invention can utilize chemical logic allowing chemical cascades to be controlled using microencapsulated reagents. Chemical logic allows a chemical pathway to be chosen because of a previously existing condition in the cascade. This is equivalent to an if-then statement in a computer program. For example, if initial condition A is true, then microcapsule 1 dissolves, releasing its reagent and microcapsule 2 does not dissolve. If initial condition B is true, then microcapsule 2 dissolves, releasing its reagent, and microcapsule 1 does not dissolve. This is equivalent to the control logic: if A then release release reagent 1; If B then release reagent 2. To illustrate this automated chemical cascade logic, consider a system where the shell of microencapsulated reagent A will only dissolve in an acidic pH solution. Microencapsulated reagent B will only dissolve in a basic pH solution. This built in intelligence in the automated cascade allows branching of the chemical cascade, meaning decisions are made and paths are followed based on what microencapsulated shells sense.

The present invention can be used for chemical tests which produce a color change, or the change of any quality perceivable by human senses. The present invention can also be configured as an instrument, and detect or measure any physical effect, such as a change of temperature, pressure, volume, voltage, current, capacitance, impedance, or other physical quantity.

The present invention is useful for applications such as chemical synthesis, in which a new chemical product, including a new chemical species or a change of state is desired. For example, the present invention configured as the chemical precursors of a propellent or explosive would make a safe and reliable high energy product.

The present invention is also useful for medical applications intended to produce a therapeutic effect. An example is a bandage which contains microencapsulated reagents which react to release oxygen at a wound site.

The present invention is also useful for applications in which the mitigation of a chemical or biological compound is desired. Mitigation is process by which a harmful chemical or a biological organism, such as nerve agents or anthrax spores, are rendered less harmful, by some chemical called an active ingredient. The active ingredient can be dispersed and delivered in a liquid or free flowing foam. The present invention can be used to deliver a microencapsulated active ingredient that has a long shelf life and can be designed to release gradually. The gradual time release is helpful because it allows all of the active ingredients to be delivered to the chemical or biological target. In the case of foam delivery, an advantage of the present invention is that the active ingredient is dispersed in the foam while in a protected microcapsule and is consequently delivered in its entirety to the target. The present invention thereby provides an effective decontamination tool.

### EXAMPLE 1

### The Present Invention Configured as a Swab for the Detection of Pesticides and Nerve Agents

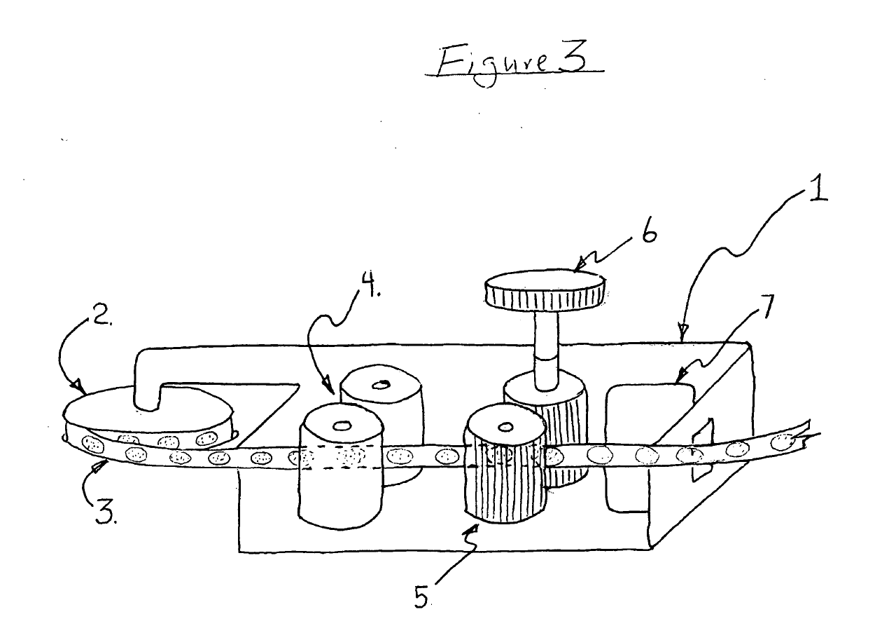

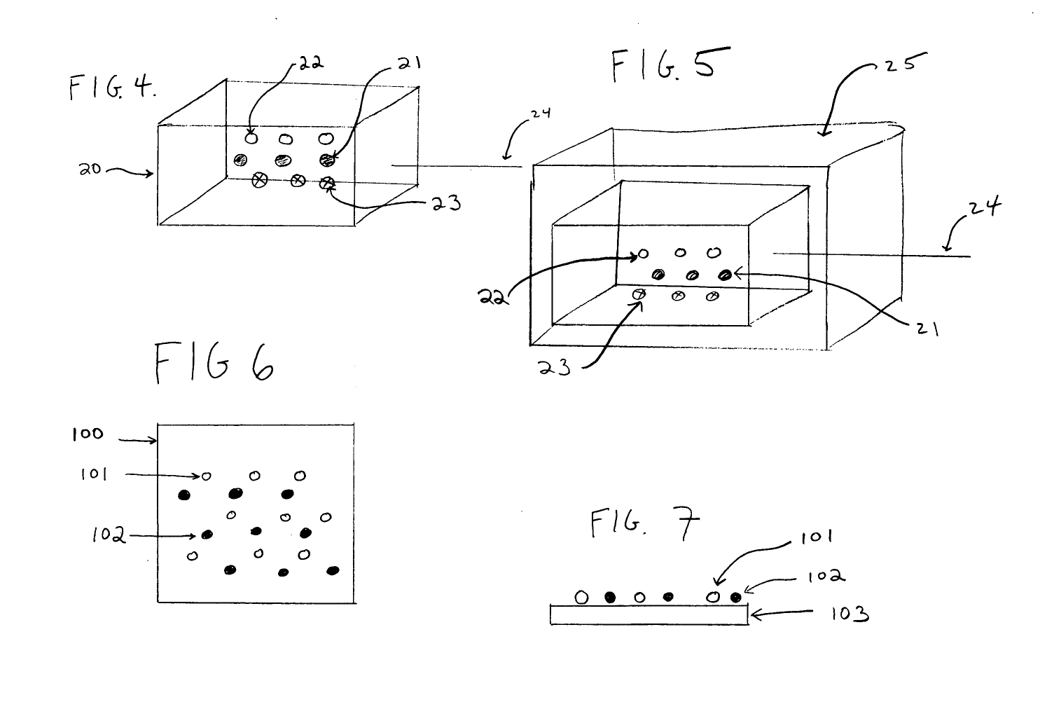

FIG. 4. shows the present invention configured as a device that measures the concentration of acetylcholinesterase inhibitors in aqueous solutions. The inhibitor in this example is called the analyte. In this example, analytes include organophosphate compounds, organosulfates, carbamates, and some venoms. Microcapsules containing Acetylcholinesterase (AChE) (21), microcapsules containing buffer (23), and microcapsules containing Indophenyl Acetate (IPA) (22) in stoichiometric ratio, are attached to a white, rigid foam structure (20). The rigid foam is attached to an elongated handle (24) which allows the operator to swipe a solution containing analyte, saturating the rigid foam structure. An optional transparent container (25) can be used to enclose the swab to contain the solution. The rigid foam provides both a support for the microencapsulated reagents, and a predefined volume. After swiping, and saturating the porous foam structure, the microcapsules containing buffer dissolve immediately. This allows the aqueous solution in the foam to come to optimal pH. AChE requires a pH of 8.0 to function optimally. Next, microcapsules which contain the enzyme AChE dissolve in buffer solution after a pre-determined interval, releasing the AChE. The AChE, buffer and any analyte that may be present incubate for a predetermined time. That interval is determined by the time it takes for the shell of the next microencapsulated reagent in the cascade, IPA, to dissolve, releasing the IPA into solution. IPA reagent then reacts in the presence of the enzyme.

This embodiment mimics the biochemical process by which these inhibitor compounds disrupt nerve transmission in organisms. Acetylcholinesterase (AChE)is an enzyme that is found in nerve synapses. Its function is to break down the neurotransmitter Acetylcholine (AChE) into two smaller molecules. If acetylcholine is not broken down into its constituents, the organism may die. Acetylcholinesterase inhibitors chemically bond to the enzyme so that the enzyme can no longer break down the acetylcholine.

This embodiment of the invention uses microencapsulated acetylcholinesterase (AChE) that interacts with molecules of acetylcholinesterase inhibitor that may be present in the sample solution. An incubation time is necessary to allow the enzyme inhibitor to bind with the enzyme, after which, a second reagent, IPA, is introduced. Indophenyl acetate (IPA) is a compound chemically similar to acetylcholine, because it is hydrolyzed by the enzyme, similar to the way that acetylcholine is broken up into two smaller molecules by the enzyme in nerve synapses. IPA is used because one of its reaction products produces a purple color. If inhibitor is not bound to the enzyme, then the enzyme hydrolyzes the indophenyl acetate, which becomes two smaller molecules, indophenoxide- and acetate. The indophenoxide- is purple colored. If an enzyme molecule is bound with inhibitor, it cannot hydrolyze IPA and no purple color is produced by that enzyme. If the test solution turns purple, this indicates intact acetylcholinesterase enzyme molecules, and therefore the absence of inhibitor. If the test solution does not turn purple, then the enzyme is no longer available, because it is bound up with inhibitor. This indicates the presence of analyte. An estimate of the analyte concentration is determined by observing or instrumentally measuring the relative purple color.

The present invention allows the colocation of microcapsules containing different reagents inside the foam structure, that are released into solution at different times.

An operator uses the device by grasping the handle and swiping the foam tip into an aqueous sample, saturating the foam. The user then waits for an appropriate time and then observes the color change, if any, in the foam. Alternatively in order to sample dry analytes on dry surfaces the present invention can contain microencapsulated working solvent, in friable shells, such as microencapsulated water. The user inserts the foam swab (20) into the transparent reaction container (25). The foam is then reciprocally plunged to release the microencapsulated solvent from friable shells, to mix the contents and develop the color.

The user compares the color of the foam to a printed color gradient. Alternatively, the user could insert the foam swab into a colorimeter that measures the color and displays the concentration of analyte.

### EXAMPLE 2

### The Present Invention Configured as a Strip Device for Detection of Pesticides and Nerve Agents

In this example, a biosensor which measures the concentration of acetylcholinestase inhibitors found on agricultural produce is described. This device utilizes the Ellman colorimetric method. Ellman's method is a colorimetric process for Acetylcholinesterase activity determination. It uses 5-thio-2nitrobenzoate (TNB-), known as Ellman's Reagent, as a chromogen producing a blue color. However if this reaction proceeds in the presence of AChE inhibitor, the color will shift from blue to yellow depending upon the amount of inhibitor present. It is this gradient in color from blue to yellow that may then be compared to a printed color gradient to indicate the quantity of inhibitor present.

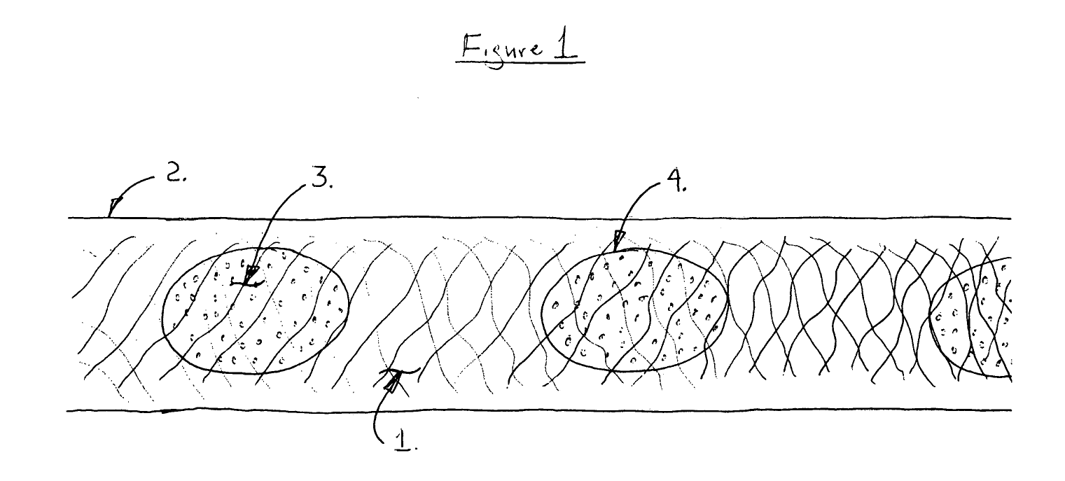

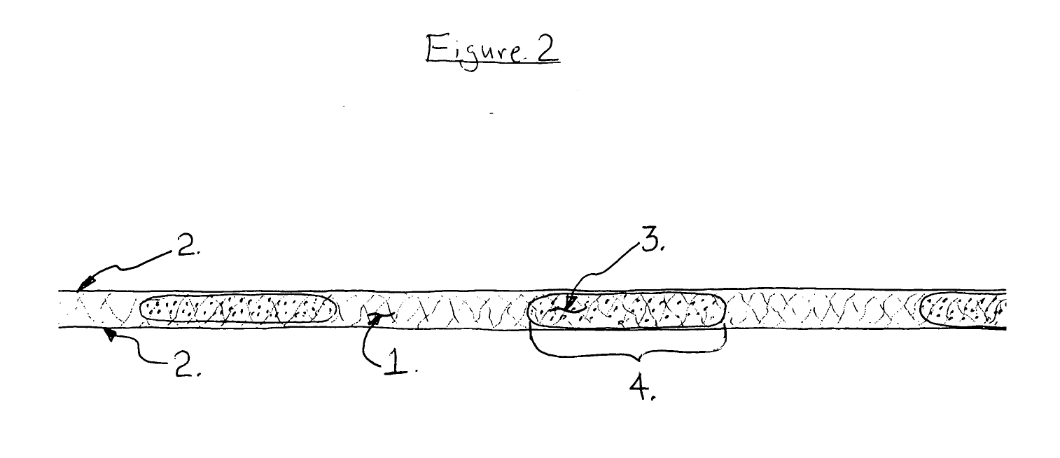

FIG. 1 shows a strip which is a sandwich composed of two outer transparent plastic layers (2), each layer 0.002 inches thick, between which is a single layer of an absorbent porous material (1) which is 0.005 thick. Microencapsulated reagents (3) are embedded in the porous layer in specific, periodic regions called reactions zones (4), each reaction zone separated from the next by 1.0 inches. The size of the reactions zones are 0.200 inch by 0.300 inch.

In use, a known volume of analyte, for example, a droplet, is placed onto the exposed reaction zone at the edge of the reagent strip between the plastic outer layers at location (13A). This initiates the chemical cascade. The clear plastic backing encases the reaction products and prevents contamination. The clear backing also allows the operator to easily observe color changes. Each discrete reaction zone is comprised of four encapsulated reagents in stochiometric quantities. Buffer, AChE and TNB, are called the stage one reagents. They are encapsulated in shells that dissolve in water. Acetylcholine (ACh) is called the second stage reagent. It is encapsulated in a friable shell. The open weave of the absorbent material (1) allows wicking of the analyte to the reaction zone where the shells surrounding stage one reagents dissolve and the reagents react.

FIG. 3 illustrates the present invention configured as a strip device (10). A bobbin (12) attached to the front part of the device, holds the reagent strip (13). The device contains one set of first stage rollers (14), which mixes the analyte and the reagents in the reaction zone on the reagent strip (13), allowing them to react. The distance between the two sets of rollers is called the incubation gap (14A). While in this gap the stage one reagents and the analyte react for a set time. A second set of crush rollers (15) are actuated by a transport knob (16). As the transport knob (16) is turned, the reagent strip (13) is transported along the length of the device (10). After the reaction zone leaves the crush rollers (15) it passes in front of a viewing stage (17). The viewing stage is a white opaque background screen, where the operator observes the color.

To make a measurement, the operator places a drop of the analyte at the drop site (13A), advances the strip past the first set of rollers (14) so that the reaction zone is located within the incubation zone (14A). The operator then waits for a predetermined time. Then the operator turns the knob (16), and advances the strip past the crush rollers (15), positioning the reaction zone in front of the viewing screen. At this point a final color is observed. The operator then compares the observed color with a printed color gradient, indicating the concentration of AChE inhibitor.

### EXAMPLE 3

### The Present Invention Configured as a Process Control Device

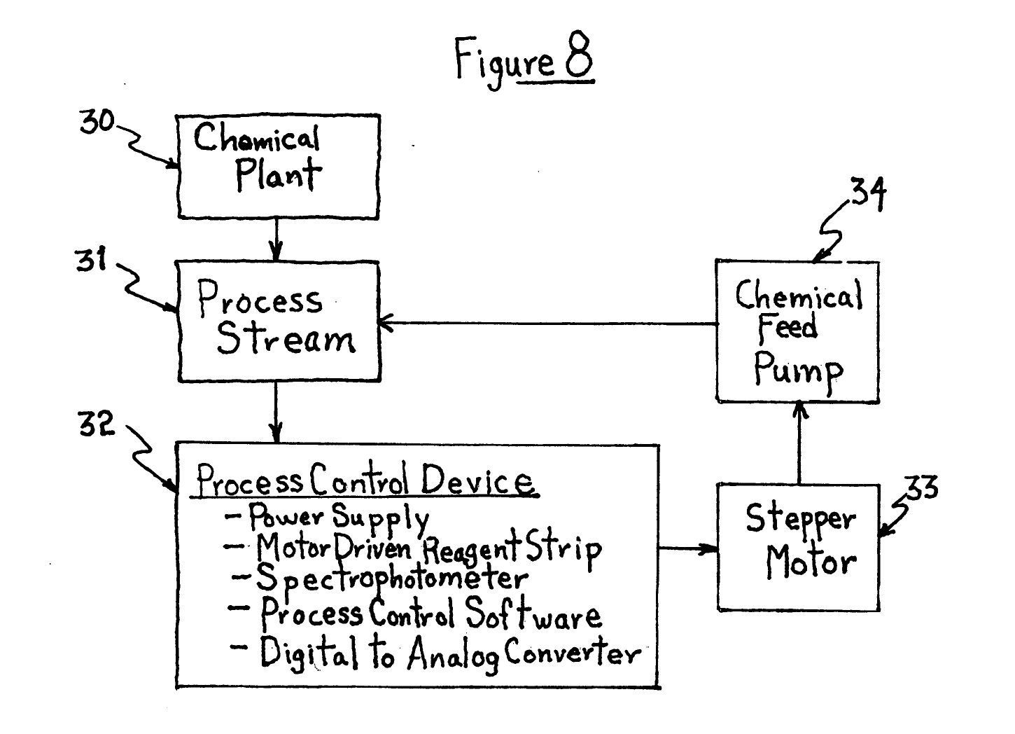

FIG 8. shows the embodiment of this invention described FIG. 3, configured as a device used for closed loop process control. A process stream (31) of a chemical plant (30) is monitored by the present invention (32). The present invention comprises a power supply, a motor driven reagent strip, a spectrophotometer or similar analytical device, process control software, and a digital to analog converter. The present invention receives a sample of analyte from the process stream. The spectrophotometer measures analyte concentration at the reaction zone. The present invention compares the measured analyte concentration to a control set point and sends a control signal to the stepper motor (33) which adjusts the stroke length of a chemical feed pump (34). The chemical output of the pump is fed back to the individual process stream (31) creating a closed control loop.

These fluid streams are found in boiler and cooling tower water, chemical plant streams, refineries, food processing plants, as well as in other applications. Prior to this invention, many analytes in process streams must be sampled, transported to an analysis laboratory, and then analyzed by laboratory personnel. The need exists for automated real time analysis and control. This invention can be adapted to any process and control stream. The device can be housed in such a way that physical and or chemical parameters, such as but not limited to, temperature, pressure, gaseous environment, electromagnetic radiation, sound, and ionizing radiation could be varied to suit any specific cascade analysis.

### EXAMPLE 4

### The Present Invention Configured as Bathroom Tissue that Detects Fecal Occult Blood

In this example a device according to the present invention that detects fecal occult blood is embedded in bathroom tissue. The analyte for this test is called occult blood because the quantities of blood can be so minute that the blood cannot be detected with the naked eye. Stool testing for blood is for the detection of colorectal cancer as well as other conditions such as recent gastrointestinal surgery, gastric (stomach) cancers, ulcers, hemorrhoids, polyps, inflammatory bowel disease, irritations or lesions of the gastrointestinal tract caused by medications (such as nonsteroidal anti-inflammatory drugs), and irritations or lesions of the gastrointestinal tract caused by stomach acid disorders, such as reflux esophagitis.

In the typical fecal occult blood test, feces are applied to a thick piece of paper attached to a thin film coated with guiaiac. A small fecal sample is applied to the film. The fecal sample is obtained either by digital rectal examination or by wiping soiled toilet tissue on the film. Both sides of the test card are peeled open, exposing the inner guaiac paper. One side of the card is marked for application of the stool and the other is for the developer fluid.

After applying the feces, one or two drops of hydrogen peroxide are dripped on to the other side of the film. A rapid blue color change indicates the presence of blood. When the hydrogen peroxide is dripped on to the guaiac paper, it oxidizes the alpha-guaiaconic acid to a blue colored quinone. When there is no blood and no enzymes called peroxidases or catalases, (which are from vegetables) present in the stool sample, the oxidation occurs very slowly. Heme, a component of hemoglobin in blood, catalyzes this reaction, giving a result in about two seconds. Therefore, a quick and intense blue color change of the guaiac paper indicates the presence of blood, a positive test result.

In this embodiment, the present invention is embedded in bathroom tissue. FIG. 6 shows a plan view of one section of bathroom tissue (100), 11.5 cm wide and 10 cm long. Attached to the surface of the paper are either friable or solvent dissolvable microcapsules containing alpha-guaiaconic acid (101) and either friable or dissolvable microcapsules containing hydrogen peroxide (102). During manufacturing of the toilet paper, the microencapsulated reagents are either sprayed or applied by another method in stoichiometric ratio to otherwise ordinary bathroom tissue.

When the user of this invention wipes the anus after defecating, microencapsulated reagent shells either dissolve or break, releasing reagent to react with blood, if present, in the feces. The bathroom tissue quickly turns blue if there is blood in the feces.

This invention allows individuals to perform their own fecal occult blood test and monitor their health for many life-threatening conditions. The manufacturing process is simple and the present invention automates the chemical reaction for the user.

Thus it will be appreciated by those skilled in the art that the present invention is not restricted to the particular preferred embodiments described with reference to the drawings, and that variations may be made therein without departing from the scope of the present invention as defined in the appended claims and equivalence thereof.

What is claimed is:

1. A microchemical reactor comprising: a chemical reactor with a volume no larger than 250 milliliters, having no moving parts, requiring no electronics or electric power, and in which microencapsulated reagents enter into multistep chemical reactions for the purpose of detecting the presence of, or measuring the concentration of an analyte, or otherwise taking part in a beneficial chemical reaction, said microchemical reactor having at least two microcapsules, each containing a different reagent; said microencapsulated reagents in a stoichiometric ratio; said microencapsulated reagents located on or in a support material; said support material can be any material that absorbs an analyte; said support material can be any material that acts as a container or delivery device; said support material providing a known volume for determination of concentration; said support material allowing co-location of microcapsules containing different reagents.

2. A microchemical reactor of claim 1 wherein the chemical reactor includes a swab comprised of any material that can be rubbed on a surface, absorbing analyte in order to determine analyte concentration.

3. A microchemical reactor of claim 1 wherein the chemical reactor includes a swab comprised of any material which can absorb an analyte, which may be a solid, liquid or gas.

4. A microchemical reactor of claim 1 wherein the reactor includes an insoluble structure of known volume, which can then be used as a sampling device, such as a foam swab, to entrain a known volume of solution containing analyte which then produces a color change indicative of the concentration of analyte.

5. A microchemical reactor of claim 1 wherein the reactor includes friable microencapsulated working solvent to allow the sampling of dry analytes on dry surfaces, said solvents are necessary for chemical reactions.

6. A microchemical reactor of claim 1 wherein the reactor comprises a continuous strip containing multiple microchemical reactors, which allows consecutive regions along the strip to interact with a known volume of test solution and allows monitoring of analyte concentrations.

7. A microchemical reactor of claim 1 wherein the reactor comprises a continuous strip containing multiple microchemical reactors, which is operated without human intervention allowing continuous monitoring of analyte concentrations.

8. A microchemical reactor of claim 1 wherein the reactor comprises a free flowing foam or liquid which can then be applied to surfaces to produce a desired chemical reaction or reaction cascade.

9. A microchemical reactor of claim 1 wherein the reactor includes a structure of any absorbent material that is applied to a human, animal or plant to produce a beneficial effect.

10. A microchemical reactor of claim 1 wherein the reactor includes microencapsulated reagents which shell material is fabricated such that electromagnetic energy can be used to open the shells.

11. A microchemical reactor of claim 1 wherein the reactor includes microencapsulated reagents which shell material is fabricated such that acoustic energy can be used to open the shells.

12. A microchemical reactor of claim 1 wherein the reactor includes microencapsulated reagents which shell material is fabricated in such a way that the presence of electromagnetic energy is detected by the opening of the shells of a microencapsulated reagent.

13. A microchemical reactor of claim 1 wherein the reactor includes microencapsulated reagents which shell material is fabricated in such a way that the presence of acoustic energy is detected by the opening of the shells of a microencapsulated reagent.

14. A microchemical reactor of claim 1 for detecting the presence of blood in feces comprising microencapsulated reagents on, or in bathroom tissue or similar materials.

15. A method for making a microchemical reactor from absorbent material for sampling an aqueous solution comprising the steps of:
a) constructing an absorbent swab having a volume no greater than 250 milliliters, wherein the swab is attached to one end of a handle;
b) locating one or more different microencapsulated reagents in stoichiometric ratio on and in the swab.

16. A method as defined in claim 15, wherein the microreactor is configured as an acetylcholinesterase inhibitor detector.

17. A method as defined in claim 15, wherein the first reagent is a microencapsulated buffer of pH 8.0 and its capsule dissolves immediately on contact with water.

18. A method as defined in claim 15, wherein the second reagent is microencapsulated acetylcholinesterase and its capsule dissolves immediately on contact with water.

19. A method as defined in claim 15, wherein the third reagent is microencapsulated indophenyl acetate and its capsule dissolves more slowly than the first two reagents, allowing acetylcholinesterase to incubate with the analyte, if present.

20. A method as defined in claim 15, wherein the absorbent swab is made out of open cell free standing foam.

21. A method as defined in claim 15, wherein said foam swab has any shape.

22. A method as defined in claim 15, wherein said foam swab is white in color or transparent so that color changes are apparent.

23. A method for making a microchemical reactor from absorbent material for sampling a dry analyte on a dry surface comprising the steps of:
a) constructing an absorbent swab having a volume no greater than 250 milliliters, wherein the swab is attached to one end of a handle;
b) locating one or more different microencapsulated reagents in stoichiometric ratio on and in the swab.

24. A method as defined in claim 23, wherein the microreactor is configured as an acetylcholinesterase inhibitor detector.

25. A method as defined in claim 23, wherein the absorbent swab is made out of open cell free standing foam.

26. A method as defined in claim 23, wherein the first reagent is a microencapsulated working solvent and buffer of pH 8.0 and its capsule is friable.

27. A method as defined in claim 23, wherein the second reagent is microencapsulated acetylcholinesterase and its capsule dissolves immediately on contact with working solvent.

28. A method as defined in claim 23, wherein the third reagent is microencapsulated indophenyl acetate and its capsule dissolves in working solvent more slowly than the first two reagents, allowing acetylcholinesterase to incubate with the analyte, if present.

29. A method as defined in claim 23, wherein the swab is inserted into a container and agitated thereby releasing working solvent from friable microencapsulated shells, creating an analyte solution.

30. A method as defined in claim 23, wherein said swab is white in color or transparent so that color changes are apparent.

31. A method as defined in claim 23, wherein said swab is swiped on a dry surface and entrains analyte on and in the swab.

32. A method as defined in claim 23, wherein after agitating foam swab in a container the swab changes color at a later time because of completion of the chemical reaction indicating presence or concentration of acetylcholinesterase inhibitors.

33. A method as defined in claim 23, wherein after completion of chemical reaction, the color of the swab is compared to a printed color standard in order to determine the presence or amount of the analyte.

34. A method as defined in claim 23, wherein after completion of chemical reaction, the swab is inserted into a colorimeter instrument to electronically determine analyte concentration.
